(19) United States
(12) Patent Application Publication
(10) Pub. No.: US 2006/0235296 A1
(43) Pub. Date: Oct. 19, 2006

(54) Contrast enhanced x-ray phase imaging

(75) Inventors: Marco Mattiuzzi, Milano (IT); Fulvia Arfelli, Trieste (IT); Ralf-Hendrik Menk, Basovizza (IT); Luigi Rigon, Trieste (IT); Hans-Jürgen Besch, Siegen (DE)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: Bracco Imaging S.p.A., Milan (IT)

(21) Appl. No.: 10/545,336

(22) PCT Filed: Feb. 10, 2004
(86) PCT No.: PCT/EP04/01213

§ 371 Date: Feb. 2, 2006

Related U.S. Application Data
(60) Provisional application No. 60/446,986, filed on Feb. 13, 2003.

(51) Int. Cl. A61B 6/00

(52) U.S. Cl. 600/431

(57) ABSTRACT

The invention refers to methods of Phase-Sensitive X-ray Imaging wherein the contrast is enhanced by use of a contrast agent selected from contrats agents usually used in other diagnostic techniques such as MRI, Ultrasound, X-ray absortion, PET and the like. Microparticulate and microbubbles are particularly preferred.

This invention refers to the Phase-Sensitive X-ray Imaging technical field. In particular the invention refers to methods of Phase-Sensitive X-ray Imaging wherein the contrast is enhanced by use of a suitably selected contrast agent.

### BACKGROUND OF THE INVENTION

X-rays are electromagnetic waves of short wavelength that can penetrate and pass trough the body due to their short wavelengths, typically between 0.01 and 1 nm. Just as a reference, the visible electromagnetic spectrum has wavelengths spanning roughly from 400 to 700 nm. When passing through matter X-rays are subjected to different types of interactions happening at the atomic level. If we assume that a number N of X-rays impinge on the front surface of a body, only a fraction of these will pass through the body and exit the back surface, the remaining part being absorbed. Each material is characterized by a macroscopic coefficient describing its ability to stop X-rays that is called “linear attenuation coefficient”, commonly indicated as μ, in [cm⁻¹] units, providing a measure of how many X-rays per unit length are stopped.

Historically, the basic principles of X-ray imaging currently in use in the today's diagnostic practice have remained essentially unchanged since Roentgen first discovery of X-rays over a hundred years ago. According to this conventional approach, X-rays pass through the body organ or tissue under examination and may exit the back surface or be absorbed by the same. The fraction that emerges is dependent upon the energy of the X-rays, the thickness of the body and the materials present in the body, i.e. tissue, bone, blood and so on.

The basic principles of conventional X-ray imaging and today's medical diagnostic systems rely on X-ray absorption as the sole source of information. Accordingly, differences in absorption produce contrast in the radiographic or tomographic images. In biological tissues calcium absorbs X-rays the most, fat and other soft tissues absorb less, air in lung absorbs the least, and are accordingly recorded in white, grey and black on the X-ray image, respectively. Optimal results are obtained only in distinguishing between hard and soft tissues while the distinction between different kind of soft tissues showing slight differences in density and composition is almost impossible.

By using the above principle some contrast agents are administered in order to increase the opacity of certain tissues so providing more contrasted images thereof. The imaging efficacy of these compounds is strictly related to their linear attenuation coefficient μ_{c} and to their total amount present in the tissue, for instance expressed in mL/cm³. Giving these two constraints, only compounds comprising heavy atoms (typically iodine atoms) can successfully be used, and only if administered in conspicuous amounts.

On the other hand, X-rays are actually waves with amplitude and a phase which can change as waves pass through matter and can both be measured.

The traditional imaging approach does not exploit at all phase related information that is conversely utilized by other “phase-sensitive” imaging techniques (PSIT), that only rely on refraction of X-rays and that allow to increase and/or complement conventional X-ray imaging. There is more than one imaging technique exploiting phase information as a source of image contrast. In more general terms a phase-sensitive imaging is defined as a technique that uses the wave phase, φ(r,t,) and, particularly, phase changes introduced in the incident x rays on passing through the sample, as the source of contrast for the image. According to the different way of measuring φ(r,t,) said techniques could be broadly, but not only, categorized as: interferometric, diffraction enhanced imaging (DEI) (also referred to as phase dispersion imaging (PDI)) and in-line imaging (or holography) methods. In general, a phase-sensitive imaging technique is an imaging procedure that uses a direct functional form ƒ(φ(r,t)) or a differential form of any order n in space or time dⁿƒ(φ(r,t))/drⁿ or dⁿƒ(φ(r,t))/dtⁿ, or even an integral form in space or time ∫ƒ(φ(r,t))dr or ∫ƒ(φ(r,t))dt or ∫∫ƒ(φ(r,t))drdt (e.g. Fourier and/or any similar transform) or an integro-differential form of φ(r,t) as source of the images contrast, where ƒ(φ(r,t)) is any function expressing a dependence on the wave phase φ(r,t) in space and/or time, symbolized as r and t respectively.

The potential of phase-sensitive techniques can be appreciated considering that for soft tissues the phase signal can be up to 10³ higher than for absorption signal depending on tissue type and X-ray energy. This extreme signal sensitivity can, in principle, discriminate differences in material densities of the order of 10⁻⁹ g/cm³, whereas X-ray computed Tomography is reported to recognize as low as 10⁻² g/cm³ density difference for 1-2 mm resolution at reasonable radiation dose (Webb S. (ed) The Physics of Medical Imaging. 1978, Bristol).

Due to its extreme sensitivity, it is usually accepted that phase-sensitive imaging does not need the administration of contrast agents (U.S. Pat. No. 5,715,291; V. N. Ingal and e. A. Beliaevskaya, J. Phys. D: Appl. Phys., 28, 2314, 1995; V. N. Ingal and E. A. Beliaevskaya, Il Nuovo Cimento, 19, 513-520 and 553-560, 1997; T. Takeda et al., Radiology, 214:298-301, 2000).

However, a selective contrast enhancement is still necessary even when the phase contrast is used, particularly when high resolution is necessary to visualize very small malignancy and calcifications or the microvasculature in a body organ or tissue or when the diagnostic imaging of a targeted organ or tissues is desired.

There are some instances in which the administration of an external contrasting compound has been considered above cited U.S. Pat. No. 5,715,291 includes a generic suggestion on the possible selection of an optional contrast agent from a wide variety of compounds.

Very recently, a study aimed at potential candidates for contrast agents in phase-contrast X-ray imaging has been proposed. Some physiological materials composed of low-atomic-number elements such as a physiological saline solution have been tested as candidate contrast agents for the selective angiography by use of interferometric phase-contrast X-ray technique (Takeda T et al. Circulation, 105:1708. 2002). This kind of solutions acts by modifying the blood density only. Consequently, they can generate an enhanced contrast only when used in association with techniques based on the exploitation of this parameter.

Now, we have found that contrast agents conventionally used in magnetic resonance imaging (MRI), ultrasound (US), conventional X-ray, NM, Positron emission Tomography (PET), SPECT, Optical Imaging may be advantageously used in the x-ray phase-sensitive imaging.

An optimal selection of the most effective contrast agent may be performed based on the kind of the specific diagnostic information and the phase-contrast X-ray technique used.

In particular, it has been surprisingly found that the use of suitably selected contrast agents allows a selective and considerable improvement in the contrast quality and intensity with all known phase-contrast X-ray imaging techniques.

An object of the present invention is therefore the use of said contrast compounds in a method for the diagnostic imaging of a body organ or tissue by use of x-ray phase-sensitive imaging techniques.

In phase-sensitive X-ray imaging the phenomenon of refraction is of great relevance for contrast agents. When a wave pass across a boundary between two materials it is “slightly deviated” (1÷0.1 μradians) according to Fermat principle.

If the transmitted and the “slightly deviated” radiation are diffracted by a dedicated downstream crystal analyzer the different angular deviations due to differences in refraction will be amplified as differences in intensity of the diffracted x-ray.

In general, a sudden and strong variation in refractive index or in the object thickness result in a marking of the signal intensity, the object borders is where the refraction effect is typically more evident. In simple terms, the “object borders signal” or “edge-signal” is due to the interference between undisturbed and refracted (phase shifted) X-rays, that results in a loss of the X-ray intensity in the original direction. In case of a phase-object (an object having negligible absorption, transparent) and a point source, this phenomenon can be described by the following formula where the image intensity I_{x,y} in the (x,y) plane for a wave propagation along z-axis becomes

$$\begin{matrix}{I}_{x , y} ∝ 1 + \frac{λ {R}_{2}}{M} {∇}_{x , y}^{2} ϕ ( x , y , {R}_{1} , λ ) \\ = 1 - \frac{2 π {R}_{2}}{M} {∇}_{x , y}^{2} {\int }_{{z}_{1}}^{{z}_{0}} δ ( x , y , {z}^{′} ) ⅆ {z}^{′} \\ = 1 - 2 π {r}_{o} {λ}^{2} \frac{{R}_{2}}{M} {∇}_{x , y}^{2} {\int }_{{z}_{1}}^{{z}_{0}} ρ ( x , y , {z}^{′} ) ⅆ {z}^{′}\end{matrix} \qquad Eq . 1$$

where R_{1,2} are the distance from source to object and from object to detector, respectively, and M=(R₁+R₂/R₁ (U.S. Pat. No. 4,979,203).

In the case of diffraction enhanced imaging, the analyzer acts like an angular filter with a very narrow bandwidth. Photons passing through the sample are deviated by an angle that is proportional to the gradient of the real part of the refraction index. Typical values of these refraction angles for biological soft tissue are in the order of microradians or tens of microradians. The analyzer can be considered as an angular filter since the reflectivity curve of the crystal, called rocking curve, is very narrow. Typical values of the width of the rocking curve are in the range of 1-20 microradians. Therefore, the angular changes of the photon trajectory due to the gradient of the refraction index in the object plane result in intensity modulation on the detector. With this technique it is possible to simultaneously measure both the apparent absorption and the refraction image.

The method of in-line imaging is governed by Eq. 1. This method can be advantageously exploited only when the object to be imaged has negligible absorption (phase object), the coherence of the lateral source is higher that the smaller details to be imaged, and the resolution of the is spatial detector is sufficient to resolve the intensity modulations.

It has now been found that the contrast in the above mentioned phase sensitive X-ray imaging techniques may be influenced either by using an agent acting on “what is inside” the object to be imaged, hereinafter defined as “area contrast agent”, or using an agent acting on “borders” or discontinuities in said object, hereinafter defined “edge contrast agent”. Accordingly, related contrast these agents promote is hereinafter defined “area contrast” and “edge contrast” respectively.

For the nature of the technique involved, interferometric methods are based on the exploitation of area-contrast, diffraction enhanced imaging may rely on both area and edge contrast while in-line imaging is more suitable for edge contrast, because the interpretation of area contrast, even possible, is more problematic.

Edge Contrast agents are able to artificially introduce in the tissue under examination numerous and sudden discontinuities in refractive index. “Edge contrast agents” are herein also referred to as “scattering-based contrast agents”. The use of these compounds with phase-sensitive X-ray imaging techniques gives an astonishingly enhanced contrast even at low concentration.

The edge-contrast generation mechanism is not exploited at all by known contrast agents. So, the use of an edge contrast agent to enhance the contrast in phase-sensitive X-ray imaging is new and constitutes a preferred aspect of the present invention as well as a method for the phase-sensitive X-ray imaging of a human or animal body organ or tissue where a contrast enhancing agent is administered to generate an edge contrast mechanism.

Any agent endowed with edge contrast properties belong to this preferred class of contrast agents. This class preferably includes heterogeneous or particulate compounds containing micro and nano objects, including any three-dimensional object whose typical dimension range between 1 and 100 nanometres such as, but not only, nanoparticles, nanotubes, fullerenes and fullerene based structures as well as and even more preferably microbubbles or nanoparticles or microballons, previously used in ultrasound techniques.

Ultrasound agents consist of tiny microbubbles sized to pass through the smallest capillaries and they are designed to backscatter ultrasound waves to increase the strength of echoes. The microbubbles measure between 2 to 8 microns in diameter and contain either air, or perfluorocarbon gas, which has prolonged longevity due to its lower solubility. The safety of these contrast agents has been demonstrated; no serious adverse events have been reported during the clinical trials. Accordingly, more preferably the edge contrast agents include microballons, e.g. that disclosed in U.S. Pat. No. 5,840,275, U.S. Pat. No. 6,123,922, U.S. Pat. No. 6,2000,548 B1 and EP 0458745; microbubbles as disclosed in U.S. Pat. No. 5,271,928, U.S. Pat. No. 5,380,519, U.S. Pat. No. 5,445,813, incorporated herein by reference. Specific examples of contrast compounds include: perfluorocarbon-filled phospholipid microbubbles, air-filled cyanoacrylate polymer-based microspheres, dodecafluoropentane-filled microbubbles, air-filled galactose microaggregates/palmitic acid, gas-filled synthetic polymers, air-filled albumin microcapsules, dodecafluoropentane in a liquid/liquid emulsion stabilized by a surfactant, perfluoro-octyl-bromide, Perflutren; octafluoropropane (or perfluoropropane)-filled human serum albumin microspheres, perfluoro-octyl bromide, sulphur hexafluoride-filled phospholipid bubbles (Sonovue®), air-filled galactose microbubbles, air-filled human serum albumin microcapsules, perflexane-filled lipid microspheres, nitrogen-filled biospheres of human serum albumin/polylactide/gelatine, perfluorobutane-filled phospholipid bubbles, barium sulphate suspensions clays, nitrogen encapsulated in echogenic biospheres, porous microparticles such as Acusphere®, galactose micropartcle granules (Echovist®), PEG-based micelles, hollow polymeric microparticles, iron oxide particles or other iron compounds (ferumoxides, ferucarbotran, frumoxtran, PEG-feron, ferristene, ferric ammonium citrate, magnetic targeted carriers). More preferably, the edge contrast agent comprises sulphur hexafluoride-filled phospholipid bubbles and even most preferably it comprises Sonovue®.

All these echo-enhancing agents are completely invisible with conventional X-ray absorption techniques.

The edge contrast agents such as echo-enhancing agents act as a strong phase signal amplifier as they introduce many edges along the X-ray path. The phase is changed because of these artificially inserted sudden discontinuities in refractive index.

Edge contrast agents may also advantageously act either as edge contrast agents or as area contrast agents so they constitute an advantageous improvement over the contrast agent acting only as area contrast agents.

Moreover, edge contrast agents overcome the problem of producing area-contrast in images for the in-line technique where the generation of an area-contrast image is not straightforward and requires some dedicated data processing algorithms. So, while area contrast agents may advantageously enhance image contrast when used in association with interferometric methods, edge contrast agents may advantageously be used in association with all phase contrast X-ray imaging techniques.

With the edge contrast agents according to the invention the area of objects to be imaged is filled with micro/nano scaled edges that are imaged as contrasting points in the image. These contrast agents are particularly effective for in-line and DEI/PDI techniques where area-contrast is considered. These latter two techniques generate a so-called “apparent absorption” image that is actually similar to a conventional absorption image but for the presence of the so-called “extinction contrast”. This latter is caused by small angle deviations (order of mradians or of tens of μradians) that the X-ray wave undergoes when travelling through an object. Those rays that incur in these deviations are completely filtered out in the image by the crystal reflectivity function. This effect increases the image contrast with respect to a pure absorption contrast. By injecting an edge contrast agent, for the above mentioned arguments, the amount of small angle deviation (scattering) and the extinction contrast is increased. This latter effect is particularly enhanced if the contrast agent has or mimics a crystalline structure. The mimicking can be accomplished by modulating the concentration of micro/nano particulate matter so to reproduce an apparent lattice spacing of a crystalline structure.

In the cited prior art the only way that is conceived as convenient for modifying the tissue contrast relates to the dose of the administered agent: there's a correspondence between agent dose and contrast obtained.

One further object of this invention overcomes this limitation. In fact, by applying external fields we can intervene on the contrast agents to modify the parameters that make it more or less effective to phase-sensitive imaging techniques. This action can be accomplished either directly or as a consequence of the field applications, (e.g. chemical reaction induced by the external field, consequent increase in local temperature and change in local density). We here refer to all fields, electromagnetic and mechanical/sound waves (for instance, radio/micro waves, optical radiation, infrared and near-infrared radiation, magnetic gradients, ultrasonic, sub-sonic, audible fields) that change either the agent local density or that change, in a spatial-temporal way, its heterogeneity.

As an example, using a microbubble contrast agent and, with the aid of a flash of an external ultrasonic field, the microbubbles are broken. Two images are acquired, prior and post the US flash, relative to two different conditions of overall density and number of microbubbles. Another example of this can be offered by the electrooptical effect induced by a locally applied electric field that changes the refractive index; “locally” refers to both a focussed external field and to an internal field generator in the form of a macro endoscopic device or under the form of dispersed/administered micro/nano artificial dipoles. In this way, it is possible to adjust the electric field tuning the refractive index to reach a desired level of contrast with the phase-sensitive imaging techniques.

Examples of further preferred agents according to the invention include: PEG-ferron (USPIO) (iron oxide), mangafodipir trisodium salt (Mn-DPDP), ferric ammonium citrate (FAC), Gd-DOTA-dextran derivative, ferumoxides (SPIO) (iron oxide), gadobenate dimeglumine (Gd-BOPTA), ferumoxsil (iron oxide), gadoversetamide (Gd-DTPA-BMEA), Gd-labeled fibrin-binding peptide derivative, ferucarbotran (USPIO) (iron oxide), gadomer 17 (dendrimer) trimesoyl[benzene-1,3,5-tricarbonyl]core containing 2 generations of 1-lysine residues and having 24 macrocyclic Gd(III) chelates at its surface, feroxirene-ferristene (iron oxide), gadopentetic acid dimeglumine salt (Gd-DTPA), MM-Q01, gadoxetate (Gd-EOB-DTPA), motexafin gadolinium, gadomelitol, macromolecular Gd-DOTA derivative, gadozelite (Gd zeolite), gadodiamide (Gd-DTPA-BMA), code 7228 (iron oxide), gadoteridol (Gd(HP-DO3A)), ferucarbotran; magnetites (iron oxide), EP-1242, gadopentetic acid dimeglumine salt (Gd-DTPA), Gd-DTPA-DeA, B22956/1, helium, gadofosveset trisodium, ferumoxtran-10 (USPIO) (iron oxide), gadobutrol (Gd-DO3A-butrol), gadoterate meglumine (Gd-DOTA), iodixanol, Iopamidol, diatrizoate meglumine [SANO], iosarcol, iopentol, iohexol, iodine-containing micelle, sincalide, iodinated macromlecular blood pool agent, DHOG, ioxilan; ioxitol, iotrolan, iomeprol, ioxaglate, iopromide, iobitridol, nanoparticulates (N1177+PH50), iosmin, ioversol, RbCl, 2-fluoro-deoxy-glucose, Tc99m arcitumomab, Tc99m DMSA (succimer) [MLCK], I131 iobenguane (MIBG) [SCHE], radiolabeled MIDAS peptides, Ga67 citrate, citrate dextrose [BRAC], I125 albumin [DRAX], I123 iobenguane (MIBG) [SCHE], Tc99m phytate, Tc99m HDP (oxidronate) [MLCK], I123 ion-channel blockers, Tc99m mebrofenin [SCHE], Tc99m MDP (medronate) [SCHE], Tc99m MDP (medronate) [SCHE], In111 ibritumomab tiuxetan, Tc99m-labeled peptide, I123 iofetamine hydrochloride (IMP), Tc99m-labeled MAB (BW 250/183), I123 iofetamine hydrochloride (IMP), Tc99m-labeled MAB, Tc99m-labeled compound (O-1506), In111 chloride, Tc99m PYP (pyrophosphate) [BRAC], Tc99m gluceptate [BMS], Tc99m votumumab, Tc99m HSA (human serum albumin) [SCHE], citrate dextrose [DRAX], Tc99m PYP (pyrophosphate) [MLCK], Tc99m chelate, Co57 cyanocobalamin [BRAC], In111 oxyquinoline, Tc99m MAA (albumin macroaggregate) [BMS], Tc99m AA (albumin aggregate) [BRAC], Tc99m PYP (pyrophosphate) [SCHE], radiolabeled MAB, Xe133 [DRAX], I131 iodohippurate [DRAX], Tc99m DMSA (succimer) [AM], Thallous chloride [AM], Tc99m mebrofenin, Tc99m depreotide, Tc99m DTPA pentetate, Tc99m albumin colloid, Tc99m tetrofosmin, Tc99m folate-terget agent, Tc99m HIDA, Tc99m sulesomab, Tc99m HDP.

When compared with conventional contrast compounds and with results obtained by use thereof in conventional X-ray radiography and tomography, the use of contrast agents in phase-sensitive X-ray imaging according to the method of invention solves several problems, particularly:

1. the necessity of using X-ray contrast agents (XRCA) including high Z materials for absorption imaging: contrast enhanced phase-sensitive imaging according to the method of the invention does not need high Z materials;

2. the high dosage of contrast agents necessary in conventional X-ray imaging: XRCA are today administered in amounts of the order of some hundreds millilitres, wile phase-sensitive X-ray imaging requests much lower doses;

3. XRCA cannot be targeted because of low signal sensitivity. Because of very high signal sensitivity phase-sensitive X-ray imaging makes X-ray targeted imaging possible. It is, in fact, possible to “wrap” an organ with a compound so to make an “envelope” at the edges or to target a compound that “sticks” inside the organ and/or pathology to be visualized;

4. XRCA cannot be used to follow metabolism for low signal sensitivity. By the new technique the compound can be chosen as to follow metabolic changes;

5. contrast agents formulated for MR, US, NM, Optical and other modalities cannot effectively be used in X-ray imaging because of high amounts to be administered in order to be effective in stopping x-rays. The doses generally administered for MRI, NM or US imaging are conversely effective when administered with phase-sensitive X-ray imaging techniques; this further results in the advantage of exploiting two different imaging modalities with a single dose of contrast agent;

6. XRCA, when include extra-cellular-fluid (ECF) agents, can be observed only for a limited amount of time because of need of a high concentration. By the new technique, the contrast agent for a longer time because of higher signal intensity can be followed;

7. NM contrast agents can be observed only for a limited amount of time because the decay of the radionuclide. When such agents are used with PSIT, because they act as area contrast agent, independently on the radionuclide activity, they are effective for longer time;

8. in-line imaging is effective for transparent objects, much less for thick objects. The administration of transparent contrast agents this problem is overcome. In a preferred method according to the invention, gas filled micro-bubbles are used that are completely transparent to absorption;

9. the use of edge contrast agent, by amplifying the signal, allows the use of detectors having lower sensitivity than that usually required in in-line imaging;

10. for both in-line imaging and DEI/PDI techniques, only the border of the object is contrasted and detectable, visible; the interior of the object can not be detected with a comparable contrast. The introduction of micro/nano objects along the x-ray path according to the method of the invention allows the enhancement of the internal region;

11. in conventional X-ray imaging, contrast agents (CA) can not be modified after administration. On the contrary, by use of phase-sensitive imaging, the CA refractive index may be modified for example by application of an external field, ultrasound, optical, thermal, magnetic field and so on;

12. contrast agents are usually chemical compounds that have different signal response compared to the biological tissue. Once injected, conventional contrast agents cannot be modified. According to the present invention, it is conversely possible to change the refractive index of both the tissue and the agent under diagnostic visualization by the use of micro/nano actuators/devices. These devices can be actively controlled and introduced orally or by an endoscopic probe;

Further advantages of the use of contrast agents according to the method of the invention are:

1. reduction of the X-ray exposure time by increasing the signal intensity;

2. improvement of the pathological diagnostic sensitivity and specificity, possible targeting of pathologies and/or morphologies and selective imaging thereof;

3. exploitation of the “edge-contrast” effect to enhance interior of an object;

4. possibility of external control of the contrasting properties of the administered compounds by application of an external field;

5. possibility of external control of the biological tissue contrast by application of an external field;

6. possible exploitation in phase-sensitive techniques of targeted agents for PECT and SPECT that show a very low activity and that consequently cannot be advantageously exploited with PET/SPECT;

7. possibility of multimodal imaging by use of a single dose of a single contrast agents (LB).

The invention is illustrated in more detail in the following experimental section.

### MATERIALS AND METHODS

Implementation of the Analyzer Crystal System

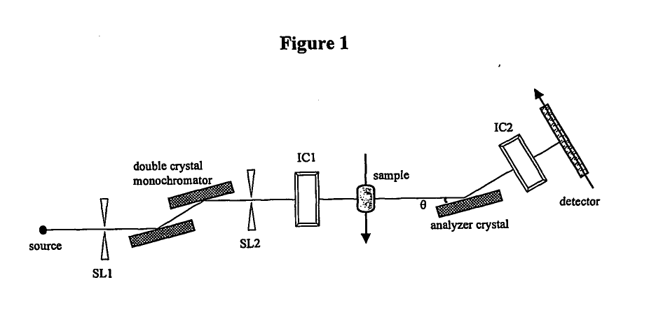

The experiment was carried out at the SYRMEP beamline at the synchrotron radiation facility ELETTRA in Trieste (Italy). The schematic layout of the experimental set-up is depicted in FIG. 1, wherein SL1 and SL2 are the slit systems, IC1 and IC2 are the ionization chambers. The source is provided by one of the bending magnets and it is vertically collimated. A monochromator based on a fixed exit Si(111) double crystal system is able to tune the energy from 8.5 keV to 35 keV. The maximum beam dimensions in the experimental hall, placed at 22 m from the source, are 150 mm horizontal by 4 mm vertical. According to the experiment the beam height can be reduced by means of a micrometric tungsten slit system positioned at the entrance of the experimental hall. The sample is located on a vertical movement stage, which can scan it through the laminar beam. A low noise CCD camera served as an imaging detector. Its active area is 29 mm×29 mm, subdivided into 2048×2048 pixels and equipped with a 40 μm thick gadolinium oxysulphide scintillator. It was placed on a second vertical translation stage that can move simultaneously to the object for the image acquisition in scanning mode.

The analyzer crystal is a flat single Si(111) crystal placed between the movement stage of the object and the stage of the detector. Its support was fixed to two Huber cradles which are moved by Berger Lahr VRDM 568/50 stepper motors. One cradle controls the Bragg angle with a precision of 1.25 10−5 degree, while the other one is used to adjust the azimuthal angle. Two custom-made ionization chambers are placed in front and behind the analyzer. From the ratio of the measured currents it is possible to evaluate the analyzer position on the rocking curve, in other words, the misalignment angle between the analyzer and the monochromator.

The Ultrasound Contrast Agents

In this study two different contrast agents normally applied for ultrasound examination have been utilized.

The Levovist® (SHU 508A, Schering AG, Berlin, Germany) contrast agent consists of granules, filled by air, composed of 99.9% galactose and 0.1% palmitic acid. Prior to use, Levovist must be reconstituted with sterilized water for injections and shaken vigorously by hand for 5 to 10 seconds. After injection of the suspension into a peripheral vein, this contrast agent leads to temporarily enhanced ultrasound echoes from the heart chambers and blood vessels. The distinct amplification of the ultrasound echo is caused primarily by micron-sized air bubbles, which are formed after suspension of the granules in water. The microspheres size is about 2-4 microns. Mediated by the palmitic acid additive, they remain stable for several minutes while in transit through the lungs and heart, and subsequent vascular bed before dissolving in the blood stream. Earlier results of clinical phase trials have demonstrated that Levovist, which was primarily designed as blood pool agent, is also promising in the characterization of focal liver tumors.

The Optison™ (FS069, Mallinckrodt Inc., San Diego, Calif.) contrast agent is an injectable suspension of microspheres composed of 1% human albumin sonicated in the presence of the inert gas octafluoropropane. Each milliliter of Optison contains 5.0-8.0×10 human albumin microspheres with mean diameter of 2.0-4.5 μm, of which 93% are smaller than 10 μm in diameter. Optison is fully manufactured before being filled into 3-mL single-use vials. No preparation of the product is required other than simply resuspending the microspheres into solution by gentle mixing. It is currently indicated for use in patients with suboptimal echocardiograms to opacify the left ventricle and to improve the delineation of the left-ventricular endocardial borders.

Levovist and Optison have been proved to be safe for use at recommended doses.

Data Acquisition Procedure and Analysis

The phantom built for Levovist contrast agent consists of a set of tubes of different size obtained drilling transversally a 2 cm slab of Plexiglas. Each element can be connected via a flexible plastic tube to a glass container filled with the contrast agent. The latter can enter in the circuit by means of a peristaltic pump so to simulate an incoming bolus of contrast agent in the vessel of an organ. In the following study the 2.2 mm diameter tube was imaged.

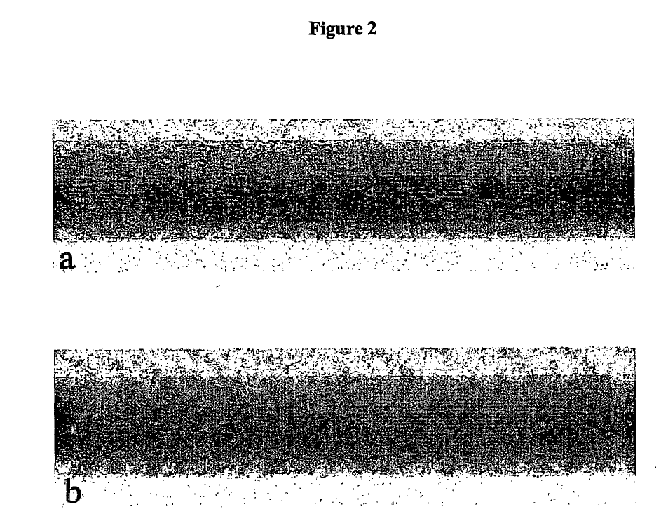

After a careful preparation of the product it was placed in the container and it was pumped inside the circuit as soon as the pump was activated remotely. The concentration was 300 mg/mL. Some images were taken while the liquid was flowing inside the phantom and immediately afterwards the pump was stopped to perform the image acquisition with the liquid stationary for comparison. No substantial difference was found in the contrast measured between the tube and the surrounding area; only some artifact due to the motion of the larger bubbles can be seen but this does not affect the overall results (FIG. 2: (a) the liquid was flowing during the image acquisition, (b) the liquid was still after stopping the peristaltic pump). Furthermore we have tested two different acquisition modes. First, the images were obtained by scanning simultaneously the phantom and the detector; secondly, the phantom and the CCD camera were both still and the vertical slits were adjusted to provide a sufficient beam height. In this case the acquisition time was much shorter (2 seconds instead of 10 seconds for the scanning mode), but we noticed a higher disomogenity in the beam mainly due to the defects in the monochromator crystals. In the scanning mode these defects are less visible since they are averaged along the scan. The results described in the section 3.1 report the images acquired in scanning mode with the contrast agent not flowing in the circuit.

The phantom built for the Optison contrast agent consists of a Plexiglas slab 2 cm thick with a rotating cylindrical tube inside. The cylinder was 8.8 mm in diameter. The rotation was essential since the microspheres in suspension in the Optison have the tendency to move to the top of the cylinder due to their lower density. With the slow rotational movement a uniform condition was achieved during the time of the image acquisition. The images were acquired only by scanning the phantom and the CCD detector through the beam because in this case the tube diameter exceeded the beam height available at the experimental station. The images were acquired in 10 seconds.

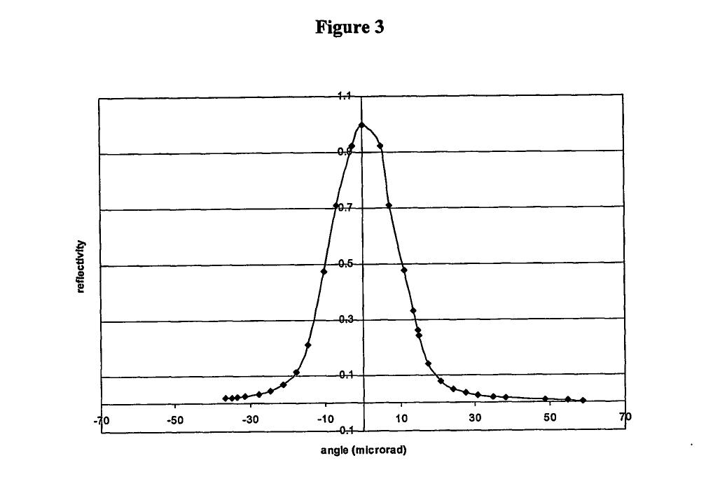

Images with both phantoms have been taken at 17 keV and 25 keV at different positions of the rocking curve. The width of the rocking curves for a Si(111) crystal at 17 keV (FIG. 3) and 25 keV were experimentally measured to be 19 μrad and 12 μrad, respectively.

For comparing the visibility and measuring the contrast in normal absorption modality a set of images have been produced without analyzer crystal but placing the phantom in direct contact with the detector.

Experimental rocking curve obtained at 17 keV as a function of the misalignment angle of the analyzer is shown in FIG. 3.

For a quantitative analysis of the visibility of the contrast and the signal-to-noise ratio (SNR) have been measured in all the images. Since the tubes filled by contrast agents have a cylindrical shape in the measurements only the central part of the cylinder was considered as a detail (with the thickness equal to the tube diameter). The following definition of contrast C was applied:

$$C = \frac{{N}_{1} - {N}_{2}}{{N}_{1}} \qquad ( 1 )$$

where N₁ and N₂ are the average counts per pixel measured respectively on the background and on the detail.

The definition of SNR was evaluated using the classic definition:

$$SNR = \frac{{ACN}_{1}}{σ ( {AN}_{1} )} \qquad ( 2 )$$

where A is the area of the detail of interest, measured in pixel number, and σ(AN₁) is the standard deviation of the counts measured in an equivalent area A in the background. In first approximation the contrast does not depend on the dose. Since the SNR depends on the dose delivered to the sample all the images have been acquired approximately at the same dose.

### RESULTS

The images have been acquired at different positions of the rocking curve of the analyzer crystal. These positions have been called far slope, slope and top in the following description. The far slope points correspond to the toes of the rocking curve where a relatively large misalignment angle between the analyzer and the monochromator has been introduced in order to achieve about 10% of the reflectivity. The angle can be positive (plus) or negative (minus). The slope represents a misalignment angle at about 50% of the reflectivity, while when the analyzer and the monochromator are perfectly aligned the position is the top of the rocking curve.

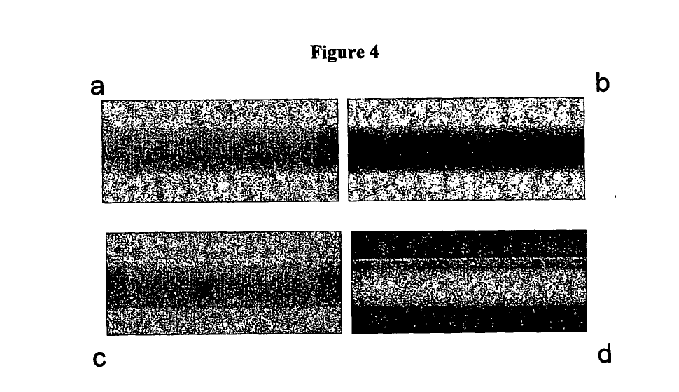

The images obtained at 17 keV are shown in FIG. 4. The absorption image, as already mentioned, is produced for comparison as a normal radiograph without the analyzer crystal (FIG. 4a). The other images are obtained at the top position (FIG. 4b), at the slope plus (FIG. 4c) and at the far slope plus (FIG. 4d). The images on the negative slope are similar to the positive one because of the symmetry of the rocking curve. A strong contrast can be noted in the image at the top and on the far slope a reverse contrast effect is evident. Here the scattering contributes significantly to the signal recorded by the detector.

The contrast and the SNR have been measured in each image and the results are summarized in Tab. 1. The contrast in the image at the top is almost 4 times larger than the contrast in the absorption image. This is due to the strong extinction effect that is added up to the normal absorption. Here, a large amount of scattering produced by the microbubbles at angle larger than the rocking curve width is completely suppressed.

At the far slope the contrast still increases (almost 10 times higher than the absorption contrast) and is negative (in Tab. 1 is reported in absolute value) due to the fact that the number of non-deviated X-rays in the background area is reduced while the scattered photons become dominant. The image at about 50% of the slope shows a lower contrast because of the presence of an inversion point on the slope where the extinction is balanced by the scattering: here the contrast would be cancelled. However in the image at the slope the inversion point was not completely reached.

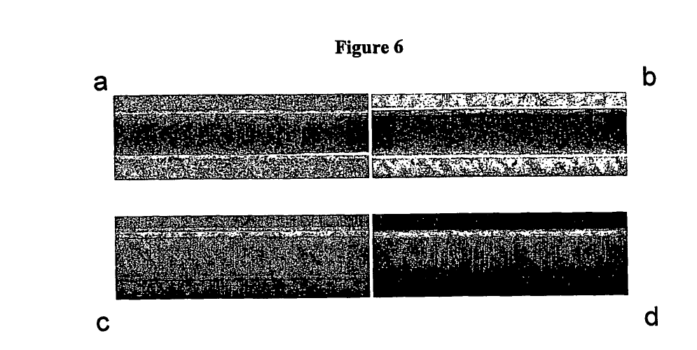

The trend of the contrast as function of the misalignment angle of the analyzer can be observed in the plot shown in FIG. 6. It should be noted that the SNR is also higher than in the absorption image, but it has a different tendency since it shows a higher value at the top than at the far slope images. The explanation comes from the lower statistics in the images at the far slope positions obtained at the same dose delivered to the phantom but only at 10% of the reflectivity of the analyzer.

TABLE 1
Contrast and SNR for images of the Levovist phantom
acquired at 17 keV at different positions of the rocking curve. The tilt
angle represents the misalignment angle between the analyzer and the
monochromator.

| | Tilt angle | Contrast | |
|---|---|---|---|
| Rocking curve point | (microrad) | (%) | SNR |
| far slope minus (8%) | −20.1 | 52 ± 2 | 140 ± 10 |
| slope minus (69%) | −7.3 | 16 ± 1 | 170 ± 10 |
| top (100%) | 0.0 | 22 ± 1 | 200 ± 10 |
| slope plus (59%) | 9.6 | 14 ± 1 | 120 ± 10 |
| far slope plus (10%) | 19.4 | 42 ± 2 | 120 ± 10 |
| absorption | — | 6 ± 1 | 63 ± 5 |

A set of images of the same phantom has been acquired at 25 keV. In this case the absorption image was in practice not visible while the contrast in the image at the top was still good (15±1%). At the far slope minus the contrast increases at 82±5% while it decreases on the slope minus close to the inversion point (8±1%).

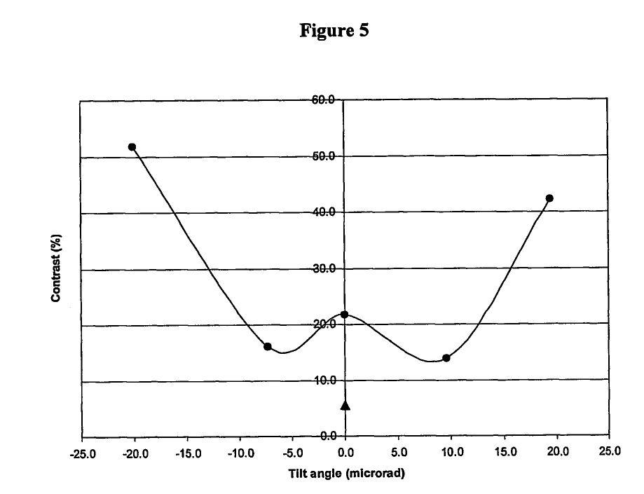

FIG. 5 shows contrast as a function of the misalignment angle of the analyzer for the Levovist phantom. The energy was 17 keV. The isolated mark represents the contrast in the absorption image.

Optison Phantom

The Optison phantom was imaged with a sequence of many different points of the rocking curve in order to appreciate better the trend of the contrast and of the SNR for small angular steps of the analyzer. The scan covered an angular range of 200 μrad centered at the top of the rocking curve and each single step was 3.5 μrad.

FIG. 6 shows images of the Optison phantom taken at 17 keV: (a) upper left: the absorption image; (b) upper right: at the top of the rocking curve; (c) lower left: at the slope of the rocking curve; (d) lower right: at the far slope of the rocking curve.

The contrast for misalignment angles smaller than few percent of reflectivity was very poor as well as the SNR. Here in practice no signal was recorded on the detector.

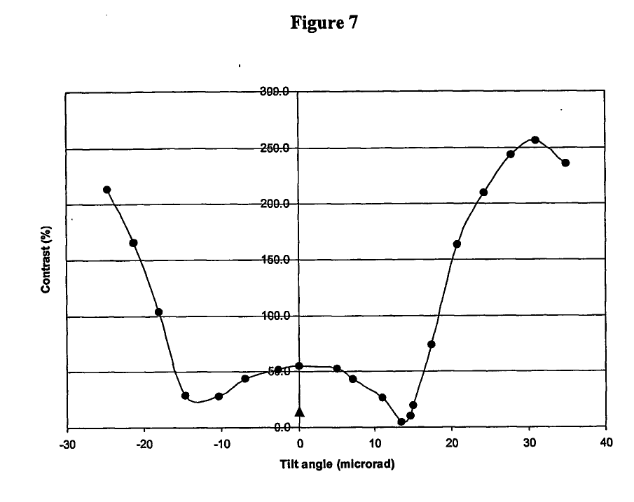

In Tab. 2 only some significant points are reported: the very high contrast at the top and at the far slope (in absolute value) positions can be compared to the poor contrast of the absorption image. The contrast at the far slope is again higher than that on the top and the SNR inverts this trend since it is greater at the top for the same reasons discussed in section 3.1. A very low contrast was obtained in the image at the slope close to the inversion point. The images corresponding to the contrasts presented in Tab. 2 are shown in FIG. 6. The trend of the contrast is shown in the graph in FIG. 7 as a function of the misalignment angle of the analyzer for the Optison phantom. The energy was 17 keV. The isolated mark represents the contrast in the absorption image.

Here the contrast behavior confirms that one already observed in FIG. 5. The contrast is again more pronounced at the far slopes than at the top reaching almost zero at the positions close to the inversion points.

Also for the Optison phantom a set of images have be acquired at 25 keV. The contrast in the absorption image was very poor around 2±1%. The signal is much higher in the image at the top with a contrast of 31±2% that decreases at the slope plus down to 5±1% growing finally at the far slope plus (56±3%). The typical trend that was found in the previous images is confirmed also in this case.

TABLE 2
Contrast and SNR for images of the Optison phantom
acquired at 17 keV at different positions of the rocking curve. The tilt
angle represents the misalignment angle between the analyzer and the
monochromator.

| | Tilt angle | Contrast | |
|---|---|---|---|
| Rocking curve point | (microrad) | (%) | SNR |
| far slope minus (5%) | −24.6 | 213 ± 5 | 250 ± 10 |
| slope minus (47%) | −10.3 | 28 ± 1 | 180 ± 10 |
| top (100%) | 0.0 | 55 ± 2 | 440 ± 10 |
| slope plus (33%) | 13.4 | 5 ± 1 | 25 ± 3 |
| far slope plus (4%) | 27.7 | 243 ± 5 | 170 ± 10 |
| absorption | — | 14 ± 1 | 110 ± 10 |

1. The use of magnetic resonance imaging (MRI), Ultrasound (US), conventional X-ray, Nuclear Medicine (NM), Positron Emission Tomography (PET), SPECT, Optical Imaging contrast agents for the preparation of diagnostic compositions for use in contrast enhanced phase-sensitive X-ray imaging.

2. The use according to claim 1, wherein said contrast agents, by introducing in the tissue under examination discontinuities in refractive index, enhance edge contrast.

3. The use according to claim 2, wherein the edge contrast agent is selected from heterogeneous or particulate compounds containing micro and nano objects.

4. The use according to claim 3, wherein said micro- or nano-objects are selected from microbubbles, nanoparticles or microballons.

5. The use according to claims 2 or 3, wherein said particulate is selected from perfluorocarbon-filled phospholipid microbubble, air-filled cyanoacrylate polymer-based microspheres, dodecafluoropentane-filled microbubble, air-filled galactose microaggregates/palmitic acid, gas-filled synthetic polymer, air-filled albumin microcapsules, dodecafluoropentane in a liquid/liquid emulsion stabilized by a surfactant, Perflutren; octafluoropropane (or perfluoropropane)-filled human serum albumin microspheres, sulphur hexafluoride-filled phospholipid bubbles, air-filled galactose microbubbles, air-filled human serum albumin microcapsules, perflexane-filled lipid microspheres, nitrogen-filled biospheres of human serum albumin/polylactid/gelatine, perfluorobutane-filled phospholipid bubbles.

6. The use according to claim 5, wherein sulphur hexafluoride-filled phospholipid bubbles are used as contrast agent.

7. The use according to claim 1, wherein the contrast agent is selected from 1 PEG-ferron (USPIO) (iron oxide), mangafodipir trisodium salt (Mn-DPDP), ferric ammonium citrate (FAC), Gd-DOTA-dextran derivative, ferumoxides (SPIO) (iron oxide), gadobenate dimeglumine (Gd-BOPTA), ferumoxsil (iron oxide), gadoversetamide (Gd-DTPA-BMEA), Gd-labeled fibrin-binding peptide derivative, ferucarbotran (USPIO) (iron oxide), gadomer 17 (dendrimer) trimesoyl[benzene-1,3,5-tricarbonyl]core containing 2 generations of 1-lysine residues and having 24 macrocyclic Gd(III) chelates at its surface, feroxirene-ferristene (iron oxide), gadopentetic acid dimeglumine salt (Gd-DTPA), MM-Q01, gadoxetate (Gd-EOB-DTPA), motexafin gadolinium, gadomelitol, macromolecular Gd-DOTA derivative, gadozelite (Gd zeolite), gadodiamide (Gd-DTPA-BMA), code 7228 (iron oxide), gadoteridol (Gd(HP-DO3A)), ferucarbotran; magnetites (iron oxide), EP-1242, gadopentetic acid dimeglumine salt (Gd-DTPA), Gd-DTPA-DeA, B22956/1, helium, gadofosveset trisodium, ferumoxtran-10 (USPIO) (iron oxide), gadobutrol (Gd-DO3A-butrol), gadoterate meglumine (Gd-DOTA), iodixanol, Iopamidol, diatrizoate meglumine [SANO], iosarcol, iopentol, iohexol, iodine-containing micelle, sincalide, iodinated macromolecular blood pool agent, DHOG, ioxilan; ioxitol, iotrolan, iomeprol, ioxaglate, iopromide, iobitridol, nanoparticulates (N1177+PH50), iosmin, ioversol, RbCl, 2-fluoro-deoxy-glucose, Tc99m arcitumomab, Tc99m DMSA (succimer) [MLCK], I131 iobenguane (MIBG) [SCHE], radiolabeled MIDAS peptides, Ga67 citrate, citrate dextrose [BRAC], I125 albumin [DRAX], I123 iobenguane (MIBG) [SCHE], Tc99m phytate, Tc99m HDP (oxidronate) [MLCK], I123 ion-channel blockers, Tc99m mebrofenin [SCHE], Tc99m MDP (medronate) [SCHE], Tc99m MDP (medronate) [SCHE], In111 ibritumomab tiuxetan, Tc99m-labeled peptide, I123 iofetamine hydrochloride (IMP), Tc99m-labeled MAB (BW 250/183), I123 iofetamine hydrochloride (IMP), Tc99m-labeled MAB, Tc99m-labeled compound (O-1506), In111 chloride, Tc99m PYP (pyrophosphate) [BRAC], Tc99m gluceptate [BMS], Tc99m votumumab, Tc99m HSA (human serum albumin) [SCHE], citrate dextrose [DRAX], Tc99m PYP (pyrophosphate) [MLCK], Tc99m chelate, Co57 cyanocobalamin [BRAC], In111 oxyquinoline, Tc99m MAA (albumin macroaggregate) [BMS], Tc99m AA (albumin aggregate) [BRAC], Tc99m PYP (pyrophosphate) [SCHE], radiolabeled MAB, Xe133 [DRAX], I131 iodohippurate [DRAX], Tc99m DMSA (succimer) [AM], Thallous chloride [AM], Tc99m mebrofenin, Tc99m depreotide, Tc99m DTPA pentetate, Tc99m albumin colloid, Tc99m tetrofosmin, Tc99m folate-terget agent, Tc99m HIDA, Tc99m sulesomab, Tc99m HDP.

8. A method of contrast enhanced phase-sensitive X-ray imaging of a body organ or tissue, which method comprises the administration to said body organ or tissue of at least one magnetic resonance imaging (MRI), Ultrasound (US), conventional X-ray, Nuclear Medicine (NM), Positron Emission Tomography (PET), SPECT, Optical Imaging contrast agent and the registration of a phase sensitive X-ray image.

9. A method according to claim 8, wherein said at least one contrast agent is an “edge” contrast agent.

10. A method according to claim 9, wherein said edge contrast agent is selected from heterogeneous or particulate compounds containing micro and nano objects.

11. The method according to claim 10, wherein said micro- or nano-objects are selected from microbubbles, nanoparticles or microballons.
